(19) United States
(12) Patent Application Publication
(10) Pub. No.: US 2003/0088888 A1
(43) Pub. Date: May 8, 2003

(54) Method of plant transformation

(76) Inventors: Alexander Vainstein, Rehovot (IL); Amir Zuker, Nes Ziona (IL)

Correspondence Address: FISH & RICHARDSON PC, 225 FRANKLIN ST, BOSTON, MA, 02110, US

(21) Appl. No.: 09/992,555

(22) Filed: Nov. 6, 2001

(51) Int. Cl. A01H 5/00; C12N 15/82

(52) U.S. Cl. 800/284; 800/294; 800/314

(57) ABSTRACT

A method for transforming a gypsophila plant with a nucleic acid of interest comprising: (i) pre-treating said gypsophila plant with a gibberellin, (ii) obtaining a plant segment from said treated plant; (iii) co-cultivating said plant segment with an Agrobacterium vector comprising said nucleic acid of interest; and (iv) selecting a transformed plant segment and regenerating a transformed plant therefrom.

### FIELD OF THE INVENTION

This invention relates to a method for transforming gypsophila plants, and to transgenic gypsophila plants.

### BACKGROUND OF THE INVENTION

The following references are referred to in the text by number:

1. Zuker A, Tzfira T and Vainstein A (1998). Genetic engineering for cut-flower improvement. Biotech Adv 16: 33-79.

2. Mol J N N Holton T A and Koes R E (1995). Floriculture: genetic engineering of commercial traits. Trends Biotech 13: 350-355.

The relationship between mankind and flowers has a very long and romantic history. In more modem times, flowers have become a highly important economic commodity and today, they are sold worldwide, with a market value of over US $30 billion. Among the ca. 20 types of major cut flowers, gypsophilas are top sellers, is accounting for a large proportion of international sales.

Native to Asia and Europe, gypsophila is one of the most important flower crops worldwide. In this economically important agricultural area, the market demand for flowers with improved traits (such as new colors, new flower forms, better fragrance, disease resistance and longer vase life) constitutes the main driving force for breeders to continually create new and more attractive varieties.

Gypsophila is a member of the Caryophyllaceae, which contains over 300 species. Gypsophila paniculata is the only species that is used as a cut flower. As one of the major contributors to the flower market gypsophila is an important target for the breeding of new varieties with novel characteristics. Yet classical breeding is strongly hampered by the essentially complete sterility of gypsophila. As a consequence, a very limited number of gypsophila varieties are sold worldwide on the flower market.

New tools for the introduction of foreign genes into plants and the growing knowledge and technology related to gene identification and isolation have enabled the specific alteration of single traits in an otherwise successful cultivar. Furthermore, such developments have enabled a broadening of the available gene pool of a given species. The application of biotechnological approaches, such as genetic engineering, to cut flowers has clearly become instrumental for the floriculture industry. However, despite the great progress and interest in gene transfer to these crops, their transformation is considered routine in only a few laboratories. For the most part, its application is still an “Art form” (1,2), Examples of cut flowers and ornamental plants which have been successfully transformed using genetic engineering methods include roses, chrysanthemum, petunia, carnation, gerbera, orchids, lisianthus, and geophytes such as tulips, lilies and gladiolus (1). However, agroscientists have been unsuccessful in developing an efficient system for transforming gypsophila plants.

### SUMMARY OF THE INVENTION

In the following specification, the term “plant segment” means a portion of plant cutting such as stems, roots, leaves etc., taken from a mature plant or seedling (young plant).

It is an object of the present invention to provide a method for transforming gypsophila plants.

It is a further object of the present invention to provide transgenic gypsophila plants.

In one aspect of the invention, there is provided a method for transforming a gypsophila plant with a nucleic acid of interest comprising. pre-treating the gypsophila plant with a gibberellin; obtaining a plant segment from the treated plant; co cultivating the plant segment with an Agrobacterium vector comprising said nucleic acid of interest; and selecting and regenerating a transformed gypsophila plant from a transformed plant segment.

The present invention provides a method for efficient transient transformation and regeneration/selection of stably transformed gypsophila plants.

The method of the invention may be used with all gypsophila species, particularly Gypsophila panictilata, Gypsophila repens and Gypsophila elegans.

The term “plant section” in this specification includes various parts of the plant which may undergo the transformation process. For example, in the case of a mature plant, a cutting may be obtained from which a stein explant or leaf may be used in the process. In the case of a seedling, leaves or stem explants thereof may be used. All of these are included in the term “plant section”.

In the case of the use of stem explants, preferably one or more of the three primary nodes are inoculated with the Agrobacterium vector. More than 62 gibberellins are known. Preferred gibberellins include GA₁, GA₃, GA₄ and GA₇. A most preferred gibberellin is GA₃.

The gibberellin may be applied to the plant by numerous methods known in the art such as, e.g. spraying or drip irrigating.

Many methods are known that can be used for transformation of gypsophila plants with the nucleic acid of interest using Agrobacterium as is well known in the art (1,2). Preferred Agrobacterium strains include Agrobacterium tumefaciens and A. rhizogenes. Examples of A. tumefaciens strains are EHA105 and AGLO.

The nucleic acid of interest can be a homologous nucleic acid (i.e. from gypsophila), a heterologous nucleic acid, or a combination thereof.

One example of a preferred embodiment of the method of the invention comprises the following steps:

1. Spraying of the gypsophila plants in the greenhouse with GA₃.

2. Transformation of gypsophila stein explants with Agrobacteria at least 7 days and preferably 15-30 days after spraying.

3. Culture of transformed explants on cocultivation medium for at least 3 days and preferably 5 days. Preferably, the co-cultivation during at least the first 2 days is in the dark, and the co-cultivation during at least the last day is in the light.

4. First selection cycle: culture of three primary nodes are incubated on T3 selection media for approximately 30 days.

5. Second selection cycle: leaves are excised from primary adventitious shoots and subcultured on B1 selection medium for approximately 20-40 days. This selection stage is optional.

6. Transfer of the putative transgenic plants to elongation/rooting medium for approximately 30-40 days.

7. Hardening of the putative transgenic plants for approximately 3 weeks.

8. Propagation under standard greenhouse conditions.

In a second aspect of the invention, there is provided a transgenic gypsophila plant transformed with a nucleic acid of interest by the method mentioned above.

The invention also includes seeds and plant parts and vegetatively-derived progeny of a gypsophila plant transformed according to the method of the invention.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1 is a graph illustrating the effect of GA₃ treatment on the transient transformation efficiency of cv. Arbel stem explants. Cuttings were collected from plants 10 days (GA10) or 20 days (GA20) after GA₃ treatment. Control plants (C) were treated with water. The results are presented as percentage of GUS-expressing stem explants out of total number of explants inoculated with Agrobacterium tumefaciens EHA105/pKIWI105;

FIGS. 2a-2g are photographs illustrating transformation and regeneration of transgenic gypsophila plants. (a) Stem explants expressing GUS 5 days after inoculation with EHA105/pKIWI105; (b) Shoot regeneration from a stem explant; (c) The chimeric pattern of GUS expression following transformation with AGLO/pCGN7001 and the first selection cycle; (d) Second selection cycle of adventitious shoots. Shoots developed from the leaf area which showed resistance to kanamycin; (e,f) Solid, non-chimeric GUS expression in adventitious shoots regenerated from leaves following the second selection cycle; (g) Transgenic plant;

FIG. 3 is a graph illustrating transient transformation frequencies of different gypsophilla cultivars. Stem explants prepared from plants treated wit GA₃ were cocultivated with EHA105/pKIWI105 for 3 days in the dark and 2 days under constant light as described in Materials and Methods. The results are presented as percentage of GUS-expressing stem explants out of total number of explants inoculated with Agrobacterium tumefaciens EHA105/pKIWI105;

FIG. 4 shows the results of PCR analysis of independent GUS-expressing kanamycin-resistant transgenic clones (1-4) and untransformed (C) cv. Arbel gypsophila plants. PCR conditions for nptII and uidA analyses were as described in Materials and Methods, below. (P) Control plasmid pCGN7001;

FIG. 5 shows a Southern blot analysis of DNA from independent transgenic (1,2) and untransformed (C) cv. Arbel gypsophila plants. Total DNA (10 μg) was digested with EcoRI (left) or HindIII (right) and hybridized with a uidA probe. (P) Plasmid pCGN7001 digested with EcoRI or HindIII; and

FIG. 6 shows a PCR analysis of independent kanamycin-resistant pAM transgenic clones (1-5) and untransformed (C) cv. Arbel gypsophila plants. (P) Control plasmid pAM.

### DETAILED DESCRIPTION OF SPECIFIC EMBODIMENTS

Materials and Methods

Plant Material

Unrooted cuttings of gypsophila (Gypsophila paniculata L.) cultivars Arbel, Pestival, Flamingo, Yokinko, New Hope and P2000 were obtained from Danziger “DAN” Flower Farm (Moshav Mishmar Hashiva, Israel). Stem cuttings with six or eight filly mature leaves (not counting the apical leaves which were not fully expanded), harvested from greenhouse-grown plants and stored for up to 2 weeks at 4° C., were used to prepare stem explants (Zuker A, Ahroni A, Shejtman H and Vainstein A (1997). Adventitious shoot regeneration from leaf explants of Gypsophila paniculata L. Plant Cell Reports, 16: 775-778; Ahroni A, Zuker A, Rozen Y, Shejtman H and Vainstein A (1997). An efficient method for adventitious shoot regeneration from stem-segment explants of gwpsophila. Plant Cell Tiss Org Cult 49: 101-106). Twenty days prior to harvesting of cuttings (unless otherwise i5 indicated), mother plants were sprayed once with approximately 4 ml. of 1 mM GA₃.

Media Composition

Murashige and Skoog basal medium (MS; Murashige T and Skoog F (1962). A revised medium for rapid growth and bioassays with tobacco tissue culture. Physiol Plant 15: 473-497) with sucrose (30 g/l) and solidified with agar (8 g/l) (basic medium), was supplemented with growth regulators and antibiotics for cocultivation with Agrobacterium, regeneration and selection of adventitious shoots, and elongation and roofing of transgenic plants. All media were adjusted to pH 5.8 prior to autoclaving (121° C. for 20 min).

For cocultivation of stem ex-plants with Agrobacterium, the basic medium was supplemented with 0.1 mg/l a-naphthalene acetic acid (NAA), 0.5 mg/l 6-benzylaminopurine (BAP) and 100 μM acetosyringone (cocultivation medium). For shoot regeneration and two-step selection of transformants, the basic medium was supplemented with 0.1 mg/l NAA and 3 mg/l 1-phenyl-3(1,2,3-thiadiazol-5-yl)-urea (TDZ) (T3, first selection cycle), or with 0.1 mg/l NAA and 1 mg/l 6-benzylaminopurine (BAP) (B1, second selection cycle). Both media were also supplemented with 300 mg/l carbenicillin and, unless otherwise state 70 mg/l (for T3) or 100 mg/l kanamycin (for B1). Elongation and rooting of transgenic shoots, following the second selection cycle, were performed on the basic medium containing 0.1 mg/l NAA, 0.1 mg/l gibberellic acid (GA), 200 mg/l carbenicillin and 70 mg/l kanamycin. All cultures were maintained in a growth room at 25±1° C. under a 16-h photoperiod using cool white light (60 μmol m⁻²s⁻¹) unless otherwise indicated.

Bacteria

Agrobacterium tumefaciens strain EHA105 (Hood E, Gelvin S, Melchers L and Hoekema A (1993) New Agrobacterium helper plasmids for gene transfer to plants. Trans Res 2: 208-218) carrying the binary plasmid pKMI105 (Janssen B and Gardner R (1989) Localized transient expression of GUS in leaf disks following cocultivation with Agrobacterium. Plant Mol Biod 14: 61-72) was used for transient transformation. AGLO (Lazo G, Stein P and Ludwig R (1991) A DNA transformation-competent Arabidopsis genomic library in Agrobacterium. Bio/Tech 9: 963-967) carrying pCGN7001 (Comai L, Moran P, Maslyar D (1990) Novel and useful properties of a chimeric plant promoter combining CaAMV 35S and MAS elements. Plant Mol Biol 15: 373-381) or pAM (Zuker A, Tzfira T, Scovel G, Ovadis A, Shklarman E, Itzhaki H and Vainstein A (2001) RolC-transgenic carnation with improved agronomic traits: quantitative and qualitative analyses of greenhouse—grown plants. J Amer Hort Sci 126: 13-18) was used for stable transformation of gypsophila.

All plasmids carried the nptII gene driven by either a nopaline synthase (NOS) promoter (pKIWI105) or the 35S promoter (pCGN7001, pAM). pKIWI105 and pCGN7001 carried the uidA gene driven by either a 35S promoter (pKIWI105) or a mannopine synthetase promoter (pCGN7001). The GUS-encoding gene (uidA) is not expressed in Agrobacterium cells carrying pKIWI105 due to the lack of a bacterial ribosome-binding site, making this plasmid suitable for transient transformation studies (Janssen and Gardner 1989). Digestion of pCGN7001 with EcoRI releases a 3.8-kb fragment containing uidA and nptII. HindIII is a unique restriction site within the T-DNA fragment of pCGN7001 (Comai et at. 1990) and pAM (Zuker et al. 2001).

Bacteria from a single colony were grown at 28° C. for ca. 20 hours in liquid LB medium (10 g/l bacto-tryptone, 5 g/l bacto-yeast extract, 5 g/l NaCl, 2 g/l glucose, pH 7.5) on a rotary shaker (250 rpm). The medium was supplemented with 100 μM acetosyringone, 50 mg/l rifampicin, and 25 mg/l gentamycin or 50 mg/l kanamycin for pCGN7001/pAM or pKIWI105, respectively. Bacteria (OD₅₅₀=0.5) was harvested by centrifugation at 10000 g for 2 min; the pellet was resuspended in liquid basic medium supplemented with 100 μM acetosyringone (OD₅₅₀=0.1 or 1.0), and file suspension was used for inoculation.

Optimization of Transient Transformation

Cuttings were rinsed with 70% ethanol, then sterilized for 10 min in 1.5% (w/v) sodium hypochlorite and rinsed three times for 10 min each in sterile water. The leaves and shoot apices of the cutting were discarded and the three primary nodes were immersed for 10 min in a bacterial (A. tumefaciens EHA105/pKIWI105) suspension (OD₅₅₀=1). Inoculated stem explants were then blotted dry and cultured in an upright position on the cocultivation medium for a period of up to 5 days. Following cocultivation, stem explants were histochemically evaluated for transient GUS expression by counting the number of GUS-expressing stem explants, as well as the number of blue spots per explant under a stereo-microscope.

Transformation and Regeneration of Transgenic Plants Tissue Culture

Twenty days prior to harvesting of cuttings, mother plants were treated with GA₃ Stem explants, prepared from these cuttings, were inoculated with bacterial (AGLO/pCGN7001 or AGLO/pAM) suspension (OD₅₅₀=0.1). During cocultivation and all consecutive steps, explants were cultured in an upright position. After 5 days of culture on the cocultivation medium (3 days in the dark followed by 2 days in light), three primary nodes were sectioned into ca. 3-mm slices and transferred to T3 medium for shoot regeneration and the first selection cycle. It should be noted that apical meristem breakage was considered undesirable. Hence, to prevent the development of non-transformed axillary shoots, all identifiable shoot apices were removed from the stem explants prior to inoculation with bacteria. After 10 days of culture, the explants were cleaned again, if needed, of the occasionally developing shoots, cross-sectioned into two halves, and transferred to fresh T3 medium. After ca. 2 additional weeks, clusters of regenerated adventitious shoots were excised from the primary stem explants. Leaves from all of the shoots of each independent cluster were pulled off and cultured on B1 medium for adventitious shoot regeneration and selection of transgenes (second selection cycle). After 10 to 12 days, new adventitious shoots emerged from the leaf basal area. These shoots were subcultured on elongation/rooting media and evaluated as to their transgenic nature.

Transfer to soil

Elongated shoots (˜2 cm in length) were rooted in glass jars with vented caps (Osmotek LTD, Rehovot, Israel) for ca. 35 days in culture. Roots were cleaned of agar and regenerated plants were transferred to pots containing peat and pumice (Solit potting soil, Soli LTD, Kiryat Malachi, Israel). After 1 week in an aeroponic fogger (Shira Aeroponics, Rehovot, Israel), plants were transferred to the greenhouse and kept under periodic mist (20 s every half-hour). Following 2 weeks of misting, transgenic plants were moved to a greenhouse where they developed and flowered normally.

Evaluation of Transformants

GUS Expression

A histochemical assay of GUS activity was performed according to Stomp (1992). Tissue samples were incubated for a few hours to overnight at 37° C. in a 0.1% (w/v) X-Gluc (5-bromo-4-chloro-3-indolyl β-D-glucuronic acid sodium salt, Biosynth Inc., Staad, Switzerland) solution containing 0.1 M sodium phosphate buffer (pH 7.0), 10 mM EDTA, and 0.1% (w/v) Triton X-100. When necessary, green tissues were bleached, after staining, by immersion in 50% (v/v) EtOH for a few hours, followed by several washes with 70% EtOH. It should be noted that no background GUS activity was detectable in any of the analyzed tissues of control plants.

Polymerase Chain Reaction (PCR) Analysis

DNA extraction, primers for uidA and nptII and PCR conditions were as previously described (Tzfira et al. 1997). The predicted sizes of the amplified DNA fragments were 0.5 kb and 0.8 kb for uidA and nptII, respectively. Amplified DNA was electrophoresed on a 1.5% (w/v) agarose gel, using Tris-borate buffer (1.3 M Tris, 0.7 M boric acid and 24.5 mM EDTA, pH 8.4). Gels were stained with ethidium bromide and photographed under ultraviolet light.

Southern Blot Analysis

DNA (10 μg) was digested with HindIII or EcoRI and electrophoresed in 1% (w/v) agarose gels. DNA was transferred to a nylon membrane (Hybond N⁺, Amersham) by capillary blotting and hybridized with ³²P-labeled uidA and nptII probes (Zuker et al. 1999). Pre-hybridization and hybridization were performed as previously described (Ben-Meir and Vainstein, 1994) at 65° C. for 3 h and 18 h, respectively. Post-hybridization washes consisted of two high-stringency washes in 0.45 M NaCl, 0.045 mM sodium citrate, 0.3% (w/v) SDS, 65° C., for 20 min each, followed by one wash in 0.15 M Nacl, 0.015 mM sodium citrate, 0.1% SDS, 65° C., for 20 min. The blots were exposed to an imaging plate (Fujix Bas 1000, Fuji, Japan) for 2-7 h. The plate was then read in an imaging plate reader (Fujix Rio Imaging Analyzer Bas 1000).

Results

Optimization of Transient Transformation

uidA (GUS) reporter gene expression was used to monitor early transformation events in gypsophila stem explants. Preliminary experiments with stem explants from cv. Arbel or other varieties (cvs. Pestival, Flamingo) not treated with GA₃, testing different wounding methods (vortexing of stem explants in the presence of glass beads, sand or carborundum particles, or poking and scratching with a needle or scalpel) yielded no transient transformation following inoculation (with or without vacuum infiltration) with Agrobacterium. In contrast, when explants were generated from cuttings obtained from plants treated with GA₃, efficient and highly reproducible transient transformation was obtained, based on both the percentage of GUS-expressing inoculated explants and the frequency of transformation events per explant,

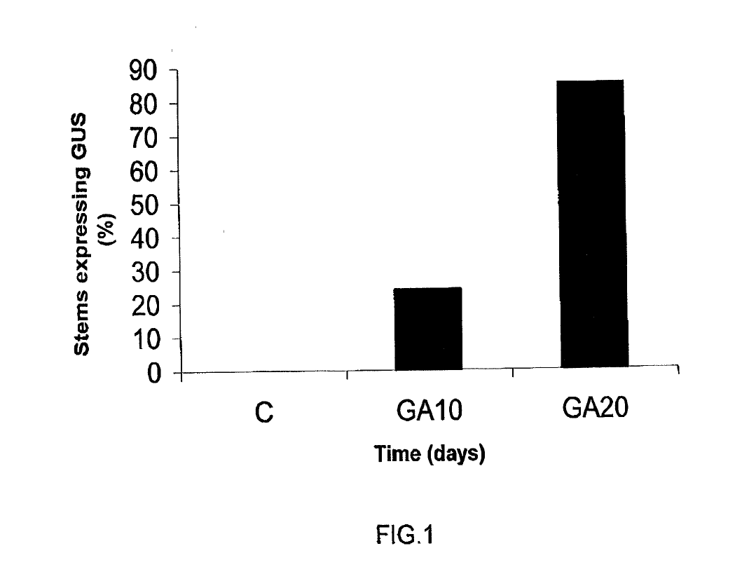

Stem explants from cv. Arbel plants 20 days after GA₃ treatment were more responsive to Agrobacterium as compared to those prepared from plants 10 days after the treatment with GA₃. Following a 5-day cocultivation of stem explants (from 20 days GA₃ treated plants) with EHA105/pKIWI105, 90% of the cv. Arbel stem explants expressed GUS (FIG. 1). Also the frequency of transient transformation events (the number of blue spots per explant) increased ca. 3 times when explants from 20 day versus 10 day GA₃ treated plants were employed, It should be noted that cocultivation with Agrobacterium for less than 5 days yielded lower transformation efficiencies.

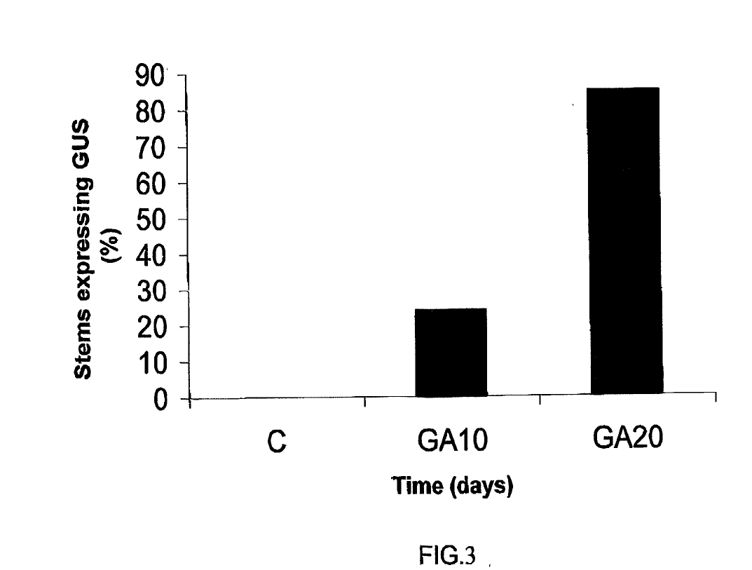

The effect of different light conditions during the 5 day cocultivation period on transient GUS transformation and regeneration efficiencies was also assessed. When cv. Arbel stem explants were cocultivated with Agrobacterium for 2-3 days in the dark then 3-2 days in the light, the efficient transient transformation was obtained (FIGS. 1,2), while high regeneration capacity of explants was retained (ca. 95% of explants yielded shoots with ca. 25 shoots per explant). To assess the suitability of the procedure to other gypsophila genotypes, transient transformation and shoot regeneration was assessed in additional varieties. In cvs. Pestival and Flamingo 60-80% of stem explants yielded shoots with ca. 10-20 shoots per explant. These cvs. were susceptible to transformation with EHA105/pKIWI105 under the aforementioned conditions and ca. 75% of the inoculated explants expressed GUS, albeit with variation in the frequency of transformation events per stem explant (FIG. 3). Similar results were obtained with the cvs. Yokinko, New Hope and P2000 (not shown).

Stable Transformation and Regeneration of Transgenic Plants

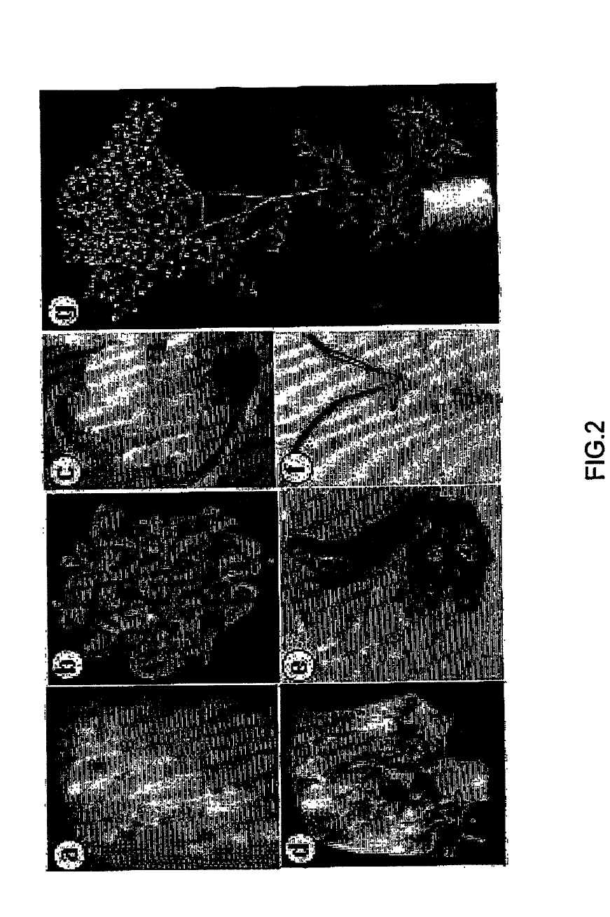

To allow for effective selection of transgenic plants on kanamycin, stable transformation of cv. Arbel was performed with pCGN7001 which carries 35S-driven nptII, rather than the NOS-driven nptII of pKIWI105 that was used in the transient transformation experiments. Inoculation of explants with bacteria at an OD₅₅₀ of 0.1-0.2 was optimal, allowing to control bacterial growth with no adverse effect on the further tissue culture and regeneration of plantlets following transfer to the regeneration/selection T3 medium. After ca. 1 month in culture following inoculation, adventitious shoot clusters, regenerated directly from sectioned stem explants, were easily scorable (FIG. 2b).

To minimize generation of putatively chimeric transgenic plants FIG. 2c) a second selection/regeneration cycle was performed. Leaves were excised from shoot clusters and cultured on B1 medium. Leaves originating from individual clusters were cultured separately for each cluster to eliminate the possibility of generating transgenes representing a single transformation event. After ca. 2-3 weeks of the second-selection cycle, 50% of the independent clusters yielded scorable shoots. These adventitious shoots (ca. 4 shoots per leaf) regenerated directly from the basal part of the leaves (FIG. 2d). In almost all of these shoots, histochemical assay revealed GUS expression throughout the tissues, with no observable chimerism (FIGS. 2e,f). To assess the overall efficiency of the two cycles of selection, only one GUS-expressing shoot per individual cluster was counted, even though 10-15 GUS-expressing shoots were usually generated from leaves of each cluster. Based on this consideration, Which allows an estimation of independent transformation events, the overall yield of the procedure was ca. 5 GUS-expressing shoots generated per 100 Agrobacterium-inoculated stem explants.

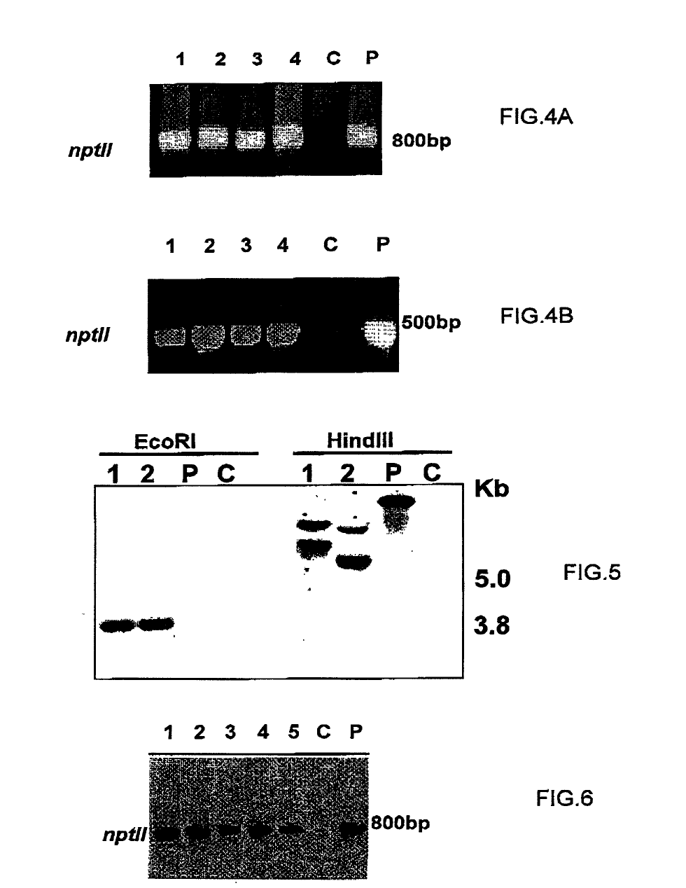

Kanamycin-resistant, GUS-expressing cv. Arbel plants (FIG. 2g) exhibited a normal phenotype when, following hardening, ca. 30 independent lines were grown to flowering in the greenhouse. The molecular analysis of transgenic plants is shown in FIG. 4 nptII and uidA PCR amplification yielded a DNA fragment of the expected size (0.8 and 0.5 kb, respectively) in all analyzed kanamycin-resistant GUS-expressing plants and not in controls.

To further confirm the transgenic nature of the generated kanamycin-resistant GUS-expressing plants, Southern blot analysis was performed (FIG. 5). Hybridization of EcoRI-digested genomic DNA with uidA probe yielded the expected 3.8 kb fragment; this fragment was not detectable in the non-transformed control line. Fragments of different sizes, as expected, were revealed following hybridization of HindIII digested genomic DNA with uidA probe (FIG. 5). Hybridization with nptII probe yielded results identical to those generated by uidA probe. These data confirm integration of the GUS-NPTII-encoding gene construct in the plant genome.

The applicability of the transformation procedure was also assessed with another binary vector pAM. Transgenic plants resistant to kanamycin were generated and grown in the greenhouse. PCR analyses of the plants, yielding expected DNA fragments, is shown in FIG. 6.

1. A method for transforming a gypsophila plant with a nucleic acid of interest comprising:
(i) pre-treating said gypsophila plant with a gibberellin,
(ii) obtaining a plant segment from said treated plant;
(iii) co-cultivating said plant segment with an Agrobacterium vector comprising said nucleic acid of interest; and
(iv) selecting and regenerating a transformed gypsophila plant from a transformed plant segment.

2. The method of claim 1 further comprising:
(v) reselecting a plant segment from said transformed gypsophila plant and regenerating a second transformed gypsophila plant.

3. A method according to claim 1 wherein said gibberellin is selected from the group consisting of GA3, GA1, GA4, and GA7.

4. A method according to claim 3 wherein said gibberellin is GA₃.

5. A method according to claim 1 wherein said Agrobacterium is A. tumefaciens or A. rhizogenes.

6. A method according to claim 5 wherein said Agrobacterium strain is EHA105 or AGLO.

7. A method according to claim 1 wherein in step (i) said plant is sprayed with said gibberellin.

8. A method according to claim 1 wherein said plant is treated with said gibberellin at least 5 days prior to obtaining said plant segments.

9. A method according to claim 8 wherein said plant is treated 15-30 days prior to obtaining said plant segment.

10. A method according to claim 1 wherein said plant segment is a stem explant or a leaf.

11. A method according to claim 10 wherein said stem explant comprises at least three primary nodes.

12. A method according to claim 11 wherein one or more of the three primary nodes of said explant are inoculated with said Agrobacterium.

13. A method according to claim 10 wherein said plant segment is derived from a seedling.

14. A method according to claim 1 wherein said plant segment is co-cultivated for at least 3 days.

15. A method according to claim 14 wherein the co-cultivation during at least the first 2 of said days is in the dark, and the co-cultivation during at least the last one of said days is in the light.

16. A transgenic gypsophila plant transformed with a nucleic acid of interest by the method of claim 1.

17. A transgenic gypsophila plant according to claim 16 of the species Gypsophila paniculata, G. peniculata or G. elegans.

18. Seeds and plant parts of a gypsophila plant according to claim 16.

19. Vegetatively-derived progeny of a gypsophila plant according to claim 16.
